(19) United States
(12) Patent Application Publication
(10) Pub. No.: US 2010/0166860 A1
(43) Pub. Date: Jul. 1, 2010

(54) Medicine for treating gastrointestinal disorder including acquired lactose-intolerance irritable bowel syndrome

(76) Inventor: James E. Lundeen, SR., Columbus, OH (US)

Correspondence Address: James E. Lundeen, Sr. , M. D., 1430 S. High St., Columbus, OH, 43207, US

(21) Appl. No.: 12/660,773

(22) Filed: Mar. 4, 2010

Related U.S. Application Data
(63) Continuation-in-part of application No. 11/283,140, filed on Nov. 18, 2005, now abandoned.
Application No. 11/283,140 is a division of application No. 10/970,164, filed on Oct. 21, 2004, now abandoned.
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.

(51) Int. Cl. A61K 31/55; A61K 9/28; A61P 1/10; A61P 1/12; A61P 1/00

(52) U.S. Cl. 424/474; 514/217

(57) ABSTRACT

A method and a medicine for treating a human having a gastrointestinal disorder that includes acquired lactose-intolerance irritable bowel syndrome are provided. The method includes administering a dose of the medicine to the human. The medicine includes ducosate sodium, tolterodine salt and imipramine salt.

### CROSS-REFERENCE TO RELATED APPLICATIONS

This application is a continuation-in-part and is filed under 37 CFR 1.53(b) and claims priority under 35 U.S.C. 120 to co-pending U.S. patent application Ser. No. 11/283,140, filed Nov. 18, 2005 which itself is a division of and claims priority under 35 U.S.C. 120 to U.S. application Ser. No. 10/970,164, filed 21 Oct. 2004, which in turn claims priority under 35 U.S.C. 119(e) to U.S. Provisional Patent Application No. 60/518,715 filed on Nov. 10, 2003; 60/518,718 filed on Nov. 10, 2003; and 60/518,719 filed on Nov. 10, 2003, the disclosures of all of which are hereby incorporated by reference in their entirety.

### BACKGROUND

1. Field of Invention

The present invention generally relates to a method and a medicine for reducing or eliminating the undesirable affects of a gastrointestinal disorder. More particularly, the invention relates to a method for reducing or eliminating symptoms of “Acquired Lactose-Intolerance Irritable Bowel Syndrome” (ALIIBS). The invention also relates to a medicine for reducing or eliminating symptoms of acquired lactose-intolerance irritable bowel syndrome.

2. Discussion of Related Art

Acquired lactose-intolerance irritable bowel syndrome (ALIIBS) may be either an acute or a chronic functional disorder of the stomach, small intestine and large bowel and is believed to affect many adults. An acute attack may last only a few days or weeks, and is not recurring over a long period of time. In contrast, a chronic affliction may last or persist for a long time, or may reoccur regularly or irregularly over a long time.

A manifestation of Acquired Lactose-Intolerance Irritable Bowel Syndrome may include abdominal pain accompanied by altered bowel function, hypersensitivity, hyperalgesia, a sense of distension, intra-luminal bleeding, and flatulence. The altered bowel function may include decreased or increased frequency of bowel movements. That is, ALIIBS may be diarrhea-predominant (D-ALIIBS), constipation-predominant (C-ALIIBS), or an alternating combination of both types. ALIIBS results from injury to gastrointestinal tract nerve network, particularly associated with acquired injury to the lumbosacral spine. Afferent and efferent nerves controlling gastric emptying time (GET) do not perform proper regulation resulting in shorter GET with premature release of gastric contents into the small bowel which cannot perform sufficient digestion and absorption of foodstuffs resulting in increased peristalisis and ultimately passing the small bowel contents into the large bowel where colonic bacteria release copious amount of gas as a by-product of bacterial digestion of otherwise undigested foodstuffs. The copious amount of gas cause distention, pain, flatulence and generally increase frequency of bowel movements.

The walls of the gastrointestinal (GI) tract have four layers: the mucosa, submucosa, muscluaris externa, and serosa. The mucosa consists of an epithelium with basement membrane (called the lamina propria), loose connective tissue, blood vessels, and lymph tissues. The submucosa contains loose connective tissue, glands, nerves, and blood vessels. The nerve fibers of the submucosa form a network or plexus called the plexus of Meissner. The muscularis externa may include two bands of smooth muscle cells, the internal layer is composed of circular smooth muscle and the external layer is composed of longitudinal fibers. Interspersed between the muscle fibers is a nerve plexus called the plexus of Auerbach. The outermost layer of the digestive tube, the serosa, is composed of a membrane of squamous epithelium.

The gastrointestinal tract has a simplified “brain”, or nerve network, in the myenteric and submucosal plexuses, which has about 100 million neurons (the enteric nervous system). Efferents in the submucosal Meissner plexus regulate secretion by intestinal glands. The efferents in the myenteric Auerbach plexus control peristalsis, which is the rhythmic contraction of circular and longitudinal muscles. Visceral sensory afferent nerve endings are located throughout the submucosa and the Meissner plexus. Cranial nerves of the parasympathetic nervous system (e.g., the vagus) convey much of the sensory information from the gastrointestinal tract. However, sympathetic afferent nerves may transmit visceral sensations of pain to the spinal cord. Sensations, motility, digestion and secretion may be controlled by nerve elements of the gastrointestinal tract. The gastrointestinal tract submucosa contains sensory afferent nerve fibers that code for pressure, temperature and pain signals. After injury to the gastrointestinal tract, especially to the mucosa, inflammation may disrupt the enteric nervous system and contribute to gastrointestinal disorders, such as acquire lactose-intolerance irritable bowel syndrome.

Current treatment options for acquired lactose-intolerance irritable bowel syndrome dietary modification to reduce intake of lactose containing food products, most notably dairy products.

With respect to diet, the patient avoids foods to which they possess a known sensitivity with respect to exacerbating the problem. A high fiber diet, either insoluble wheat bran or soluble psyllium, is almost routinely recommended, but with little if any positive benefit. For constipation-predominant acquired lactose-intolerance irritable bowel syndrome, suitable gastrointestinal stimulants, or transit time reducers include wheat bran, soluble fiber, and polycarbophil calcium. But, while the gastrointestinal stimulants are useful to reduce gastrointestinal transit time, they also may exacerbate abdominal pain and bloating, and exacerbate D-type acquired lactose-intolerance irritable bowel syndrome symptoms.

With reference to drugs or medicines for the treatment of acquired irritable bowel syndrome, none has demonstrated sufficient efficacy to be of practical benefit to a majority of patients. Drugs for the treatment of ALIIBS are treatments directed to the gastrointestinal tract. ALIIBS is a specific subgroup of IBS. The primary problem in ALIIBS is short GET due to disruption of the enteric nervous system. Irritable Bowel Syndrome (IBS) in general has no known specific cause and is widely considered to be bowel manifestations of psychological disorders and is generally treated as a mental disorder. ALIIBS is a physical disorder due to loss of control of GET which by default is short and only properly regulated with an intact enteric nervous system.

Drug treatment for IBS directed to the gastrointestinal tract includes antacids, anti-spasmodic agents, anti-diarrheal drugs, anti-inflammatory drugs such as glucocorticosteroids and NSAIDS, histamine-R2-blocking agents, antibiotics, and, in the case of ulcerative colitis, surgery to remove the affected tissues. Suitable antacids include buffered acid neutralizing agents and proton pump inhibitors. Cholestyramine is a copolymer of styrene and divinylbenzene possessing trimethylbenzyl ammonium groups, and has a somewhat limited capacity to bind bile acids. None treat the source or etiology of ALIIBS, all of these treat the symptoms.

Anti-spasmodic (anti-cholinergic) medication is recommended for IBS pain and bloating. Drugs having spasmolytic activity may be prescribed to decrease intestinal motility. U.S. Pat. Nos. 4,611,011, 4,701,457, and 4,745,131 disclose a series of amidinoureas which reduce intestinal motility and are useful for treating irritable bowel syndrome. 1-Azabicyclo[2-2-2]octan-3-yl-2-aryl-3-azacyclo-2-hydroxy propionates and their quaternary salts, which possess antispasmodic activity and are useful for treating irritable bowel syndrome, are disclosed in U.S. Pat. No. 4,843,074. Calcium channel antagonists exhibit muscle relaxing and antispasmodic activities. A series of substituted imidazolyl-alkyl-piperazine and diazepine derivatives, disclosed in U.S. Pat. No. 5,043,447, are calcium channel antagonists and may be useful as antispasmodics for treating irritable bowel syndrome. U.S. Pat. No. 4,877,779 discloses 2-Aminomethylalkynylalkyl-1,3-dithiane derivatives with calcium-channel blocking activity and potentially similar uses. Some triazinone derivatives having spasmolytic activity for treating irritable bowel syndrome are disclosed in U.S. Pat. No. 4,562,188.

In addition to antispasmodic agents, compounds with other activities have been disclosed which may relieve the symptoms of irritable bowel syndrome. Anti-diarrheal agents, such as loperamide, diphenoxylate, and codeine phosphate, have been used to treat IBS. Unfortunately, such agents may exacerbate the constipatory phase of the disease and are ineffective in treating the additional symptoms associated with IBS, such as abdominal pain. They are, therefore, of little practical long-term benefit. Other anti-diarrheals include anti-cholinergics and smooth muscle relaxants, such as cimetropium bromide, pinaverium bromide, octilium bromide, trimebutine, and mebeverine.

U.S. Pat. No. 4,239,768 discloses a series of arylimidazolidinylidene ureas which decrease the sensitivity of the bowel to distension and thereby reduce irritable bowel symptoms. U.S. Pat. No. 4,970,207 discloses some benzodiazepine derivatives which are cholecystokinin antagonists, and which may be useful for the treatment of irritable bowel syndrome. Anti-spasmodics, anti-diarrheal preparations, analgesics and the like have been used, but even if they are effective, long-term treatment is precluded by problems such as development of tolerance, toxicity, or abuse potential.

Non-selective excitatory opioid receptor antagonists have been identified as central nervous system treatments that affect the symptoms associated with irritable bowel syndrome. Non-selective excitatory opioid receptor antagonists include tricyclic antidepressants, such as amitriptyline, imipramine, and doxepin, and have been used to treat irritable bowel syndrome. These opioid receptor antagonist may be effective due to the neuromodulatory and analgesic properties of these compounds, which are independent of their psychotropic effects. The non-selective nature of the tricyclic antidepressants results in affectation of all five of the recognized muscarinic receptors and may cause undesirable side effects.

Manufacturer's recommended dosages of imipramine pamoate may be modified as necessary by response and evidence of intolerance. The manufacturer's recommended dosages include initial adult dosage for outpatients starting at 75 mg/day, which may be increased to 150 mg/day—the level at which the optimum response is usually obtained for anti-depression treatment. Also for anti-depression treatment, manufacturer's recommended dosages for hospitalized patients are to start at an even higher dose of 100-150 mg/day—and the dosage can be raised as high as 300 mg/day. Elderly patients and children are stated to likely respond to a dosage of 25-50 mg/day.

Selective excitatory opioid receptor antagonist naturally have been studied in an attempt to decrease or eliminate the undesirable side effects caused by the non-selective nature of the above non-selective excitatory opioid receptor antagonist. For example, nalmefene glucuronide has been used as a treatment for constipation-predominant IBS. Patients receiving the composition reported a decreased transit time and increased stool frequency. Unfortunately, nalmefene glucuronide did not reduce abdominal pain or bloating, and stool consistency was not improved.

U.S. Pat. No. 5,512,578 discloses co-administration of a selective excitatory opioid receptor antagonist with a bimodally-acting opioid agonist may enhance analgesic potency, and reduce tolerance and dependence liability. Such selective excitatory opioid receptor antagonists include, when administered at appropriately low doses, naloxone, naltrexone, etorphine, and dihydroetorphine. The selective excitatory opioid receptor antagonists attenuate excitatory, but not inhibitory, opioid receptor functions in nociceptive (pain) pathways of the peripheral and central nervous systems. As a result, symptoms associated with activation of excitatory opioid receptors, such as anti-analgesia, hyperalgesia, hyperexcitability, physical dependence and/or tolerance effects may be increased.

U.S. Pat. No. 6,664,270 discloses a method and composition for treating irritable bowel syndrome using a polyamine material. Unfortunately, the composition related to diarrhea-predominant acquired lactose-intolerance irritable bowel syndrome IBS, had undesirable side effects, and was not sufficiently efficacious in the treatment.

In spite of the many treatments, compositions and methods used to reduce or eliminate symptoms associated with gastrointestinal disorders, such as irritable bowel syndrome, no suitable long-term efficacious treatment or preventative has been identified. It would be desirable to have a medicinal composition or medicine having improved properties for the treatment of acquired lactose-intolerance irritable bowel syndrome. It also would be desirable to have a method for the treatment of acquired lactose-intolerance irritable bowel syndrome. ALIIBS is a specific subgroup of IBS in general.

### SUMMARY

The present invention relates to a method and a medicine for treating a human having a gastrointestinal disorder including Acquired Lactose-Intolerance Irritable Bowel Syndrome (ALIIBS). The medicine includes a salt of imipramine, Hcl or pamoate salt, Tolterodine salt and the specific stool softener, ducosate sodium. The method includes administering a dose of the medicine to the human.

In an aspect of the invention, invention relates to a process that includes interacting with muscarinic receptors in the human to reduce or eliminate at least one symptom caused by or associated with chronic acquired lactose-intolerance irritable bowel syndome. The process further includes emulsifying oil and water into fecal matter using the surfactant to soften the stool of the human.

### BRIEF DESCRIPTION OF THE DRAWINGS

none, not applicable.

### DESCRIPTION OF PREFERRED EMBODIMENTS

The present invention generally relates to a method of treating a disorder of the gastrointestinal (GI) tract with a medicinal composition, and the medicine. As used herein, a chronic condition refers to a condition that lasts for a substantial period or long time, and in some instances a chronic condition may not have an endpoint. Furthermore, chronic conditions may be continuous or recurring, and may reoccur regularly or irregularly. Gastrointestinal tract disorder includes acquired lactose-intolerance irritable bowel syndrome. Unless specified or the context dictates otherwise, acquired lactose-intolerance irritable bowel syndrome includes constipation-type acquired lactose-intolerance irritable bowel syndrome (C-ALIIBS), diarrhea-type acquired lactose-intolerance irritable bowel syndrome (D-ALIIBS), and alternating C-ALIIBS and D-ALIIBS. Constipation is intended to broadly include a reduced frequency of bowel movements up to and including obstipation. Diarrhea is intended to broadly include both a medical practitioner's definition—an increased frequency of bowel movements, and a lay person's definition—liquid or fluid stool that causes difficulty of continence. A medicinal composition (“medicine”) is a substance administered in the treatment of disease; a remedial agent; and/or a remedy. An efficacious amount is an amount greater than zero that has a desired or desirable effect.

A method according to embodiments of the invention includes administering a dose of the medicine to a patient suffering from or presenting symptoms associated with a gastrointestinal disorder, such as acquired lactose-intolerance irritable bowel syndrome. The medicine is described below, as is dosage information. The acquired lactose-intolerance irritable bowel syndrome may be a result of a nerve injury. The nerve injury may be, for example, a spinal nerve injury, spinal cord injury, or pelvic nerve injury, affecting enteric nerve function.

In one embodiment, the medicine includes a salt of imipramine, Tolterodine salt and the specific stool softener ducosate sodium which softens as well as lubricates the stool. The medicine according to embodiments of the present invention may be used to treat C-ALIIBS, D-ALIIBS, and alternating type acquired lactose-intolerance irritable bowel syndrome. The imipramine salt (that may reduce stool frequency) is present regardless of infrequent stools, and ducosate sodium (that may increase stool frequency) is present regardless of frequent stools, Tolterodine salt is present in all preparations.

Ducosate sodium as used herein is distinguished from laxatives. Laxatives may include bulk, osmotic and stimulant-type. Bulk laxatives may include soluble and insoluble fiber. Soluble fiber may include psyllium husks and is commercially available as METAMUCIL from Procter & Gamble Inc. (Cincinnati, Ohio). Insoluble fiber can include wheat bran. Osmotic laxatives are not absorbed and function by pulling water into the colon via osmotic action (e.g., magnesium hydroxide, such as PHILLIP'S MILK OF MAGNESIA, which is commercially available from Bayer Corporation (Pittsburgh, Pa.)). Stimulant laxatives interfere with absorption of water from the colon lumen and motility of fecal material there through.

By way of contrast, a stool softener acts to emulsify water and/or oil into fecal matter and thus soften the consistency. A fecal lubricant acts by lubricating the fecal matter and allowing it to pass though the colon with a reduced amount of friction. Suitable stool softeners include surfactants, such as anionic surfactants. Other suitable surfactants include nonionic surfactants, cationic surfactants, and amphoteric surfactants. In each embodiment, the stool softener includes bis(2-ethylhexyl)sulfosuccinate sodium salt (“docusate sodium”), which is commercially available from Purdue Phama L. P. (Stamford, Conn.) as COLACE. Other suitable metal salts of sulfosuccinate may be useful, and the metal may be potassium, calcium and the like.

All or a portion of the medicine may be in the form of a pill, capsule, gelcap, a coated or chewable tablet, a chewable gum, an ingestible liquid admixture, transdermal patch, an inhalable powder or mist, an enema or suppository, an intravenous solution or an intramuscular injectable liquid.

Administration of the dose may include selecting an entry method or application based on the form of the medicine. For example, if imipramine and Tolterodine salt are a gelcap, and sodium docusate is an ingestible liquid, the imipramine and Tolterodine salt may be swallowed and the sodium docusate may be imbibed or drank. In one embodiment, imipramine, Tolterodine salt and sodium docusate may be combined or commingled in a single capsule.

In one embodiment, imipramine, Tolterodine salt and sodium docusate may be combined or commingled as portions contained in a plurality of capsules. The portions may be fractional amounts of the total daily dose.

Similarly, the total daily dosage amount may be controlled by selecting portions containing fractional dosage amounts.

The number of fractional doses or portions taken per day may be adjusted to correspond to preselected factors. Such factors may include, for example, seasonal changes (e.g., dehydration, being more prevalent in summer months, may result in a temporary amelioration of fecal incontinence), aging, the natural course of the gastrointestinal disorder, stress inducing situations, and others that may affect the occurrence or severity of symptoms of the gastrointestinal disorder.

Naturally, in other embodiments (not shown) the imipramine, Tolterodine salt and stool softener portions may include dosages having differing amounts for different total daily dosages, may have differing numbers of doses for the same or different total daily dosages, and may have doses that include the imipramine, Tolterodine salt and the stool softener in a single form (such as a pill containing the imipramine, Tolterodine salt and ducosate sodium).

Other embodiments according to the invention may have the imipramine, Tolterodine salt and/or the ducosate in a form other than pill, gel cap, and the like, and may not be amenable to blister packaging. Suitable packaging may then be selected based on the form of the imipramine, Tolterodine salt and ducosate sodium, and whether the imipramine and Tolterodine salt and the ducosate sodium are admixed or physically separate.

With reference to forms of the medicine other than those discussed above, the ingestible liquid admixture may be administered in pre-measured amounts. The transdermal patch, the chewable gum, the intravenous solution, or the intramuscular injectable liquid, and the oral and/or nasal inhaler (for the inhalable powder or mist) may be used to deliver the imipramine, Tolterodine salt, while the ducosate sodium may be administered via a different method. The enema or suppository may contain the ducosate sodium and may be administered in a conventional manner. For orally administrable embodiments in which at least one component or portion of the medicine is taken orally, masking agents may be used. For example, edible carriers, such as food, may be used to enhance palatability of the medicine or medicine component. In one embodiment, the food is selected to have a pharmacological effect. For example, prune juice has a known tendency to increase bowel movement frequency, and this tendency may be factored into the dosage amounts for the medicine or medicine components.

Embodiments will contain 5 mg imipramine, 4 to 8 mg Tolterodine salt and 100 mg ducosate sodium.

The processes and embodiments described herein are examples of compositions, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other compositions, systems and methods that do not differ from the literal language of the claims, and further includes other compositions, systems and methods that are equivalent to, or have insubstantial differences from, the literal language of the claims.

What is claimed is:

1. A medicinal composition for treating a human having a gastrointestinal disorder comprising acquired lactose-intolerance irritable bowel syndrome, the composition comprising:
imipramine salt 5 mg, Tolterodine salt 4 mg to 8 mg ducosate sodium 100 mg.

2. The composition as defined in claim 1, containing 4 mg of Tolterodine salt, wherein the acquired lactose-intolerance irritable bowel syndrome is constipation type.

3. The composition as defined in claim 1, containing 8 mg of tolterodine salt, wherein the acquired lactose-intolerance irritable bowel syndrome is diarrhea type.

4. The composition as defined in claim 1, containing 6 mg of tolterodine salt, wherein the acquired lactose-intolerance irritable bowel syndrome is alternating constipation type and diarrhea type.

5. The composition as defined in claim 1, wherein the gastrointestinal disorder is a result of a nerve injury.

6. The composition as defined in claim 5, wherein the nerve injury is a spinal nerve injury, spinal cord injury, or a pelvic nerve injury affecting the enteric nervous system.

7. The composition as defined in claim 1, wherein at least a portion of the medicine is in the form of a pill, capsule, gelcap, an ingestible liquid admixture, transdermal patch, an oral or nasal inhalable powder or mist, an enema or suppository, a coated or chewable tablet, a chewable gum, an intravenous solution, or an intramuscular injectable liquid.

8. A treatment kit for a human having a gastrointestinal disorder comprising acquired lactose-intolerance irritable bowel syndrome, the kit comprising:
a plurality of doses of a medicine, the medicine comprising:
imipramine 5 mg, and tolterodine salt 4-8 mg and ducosate sodium 100 mg

9. The kit as defined in claim 8, further comprising an instruction set comprising directions for administering the medicine, the instruction set comprising dosage amounts, dosing schedules, directions for varying dosages, or combinations of two or more thereof.
